(19) United States
(12) Patent Application Publication
(10) Pub. No.: US 2005/0059692 A1
(43) Pub. Date: Mar. 17, 2005

(54) Process for the preparation of 17beta-N-[2,5-bis(trifluoromethyl)phenyl] carbamoyl-4-aza-5-alpha-androst-1-en-3-one

(75) Inventors: Manne Satyanarayana Reddy, Hyderabad (IN); Gudipati Srinivasulu, Hyderabad (IN); Katkam Srinivas, Secundarabad (IN); Komati Satyanarayana, Gangadevipadu (IN); Mayur Devjibhai Khunt, Mendarda (IN)

Correspondence Address: Janet I. Cord;Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/885,501

(22) Filed: Jul. 6, 2004

(30) Foreign Application Priority Data
Sep. 9, 2003 (IN) 719/CHE/2003

(51) Int. Cl. A61K 31/473; C07J 73/00

(52) U.S. Cl. 514/284; 546/61

(57) ABSTRACT

The present invention relates to a process for the preparation of Dutasteride, which is chemically known as 17β-N-[2,5-bis (trifluoromethyl) phenyl] carbamoyl-4-aza-5-α-androst-1-en-3-one and can be represented by Formula (I).

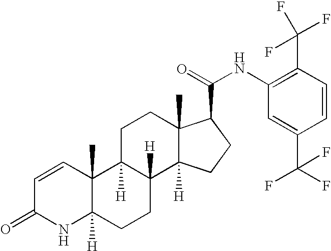

### FIELD OF THE INVENTION

The present invention relates to novel and simple process for the preparation of Dutasteride, which is chemically known as 17β-N-[2,5-bis (trifluoromethyl) phenyl] carbamoyl-4-aza-5-α-androst-1-en-3-one and can be represented by Formula (I).

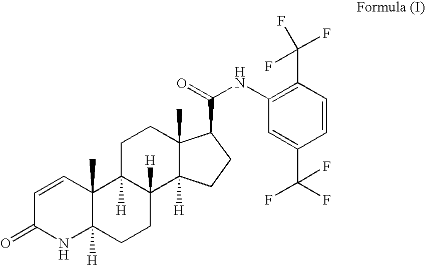

Dutasteride is useful in the treatment of androgen responsive and mediated diseases.

### BACKGROUND OF THE INVENTION

U.S. Pat. No. 5,565,467 discloses a process for the preparation of Dutasteride, which comprises the dehydrogenation of 17β-N-(2,5-bis (Trifluoromethyl) phenyl) carbamoyl-4-aza-5α-androstane-3-one (Formula Ia) by 2,3-dichloro-5,6-dicyano-1,4-benzoquinone and bis(tri methylsilyl)trifluoroacetamide in dioxane as solvent and the resultant solid is crystallized from a mixture of ethyl acetate-heptane in a ratio of 1:1 v/v. The chemical process depicted as bellow (Scheme 1)

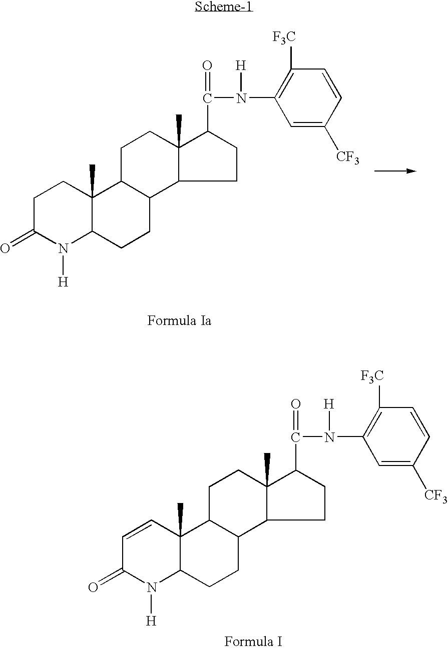

Alternatively, Dutasteride (Formula-1) can prepared from 3-oxo-4-aza-5α-androst-1-en-17β-carboxylic acid of Formula II (Rasumussion, G. H. et al., J.Med.Chem, 29, 2298(1986)) through the acid halide intermediate of Formula IIa. Acid chloride reacted with the 2,5-bis (tri fluoro methyl) aniline to form a Dutasteride.

The chemical process depicted as bellow (Scheme 2)

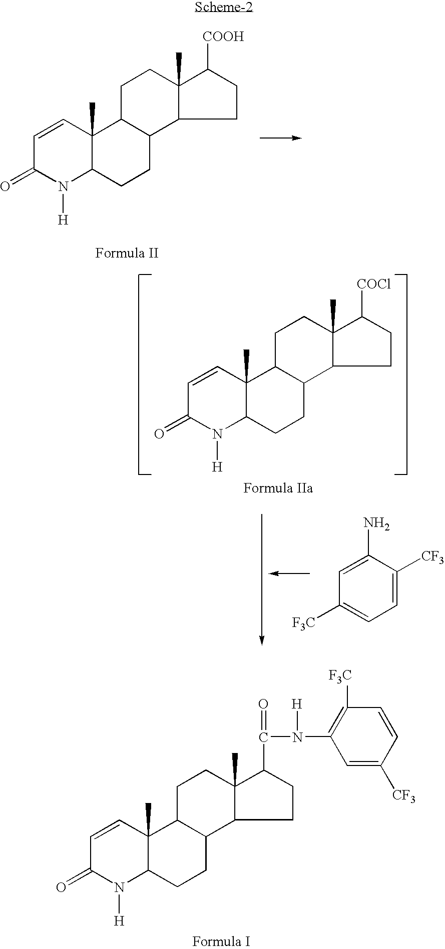

The process disclosed in prior art references have disadvantages like high raw material cost, poor yields and not suitable for scale up activities.

Hence, the object of the present invention is to provide process for the preparation of Dutasteride, which is simple, cost effective, eco-friendly, and commercially suitable process by overcoming the problems encountered in the above prior art process.

### SUMMARY OF THE INVENTION

The present invention provides novel and simple process which involves condensation between 3-oxo-4-aza-5α-androst-1-ene-17β-carboximide of Formula III and 2-Iodo-1,4-bis (trifluoromethyl) benzene of Formula IV, which can be represented by the (Scheme3)

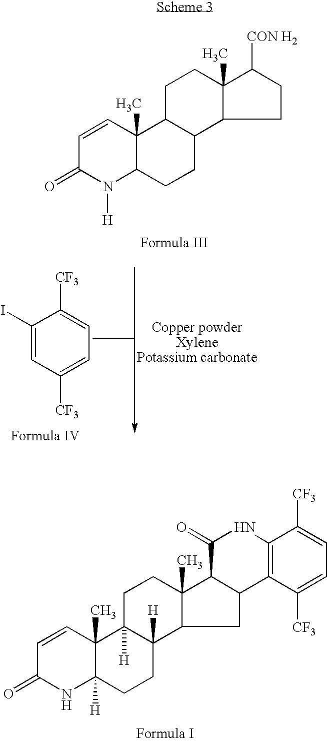

The starting compound 3-oxo-4-aza-5α-androst-1-ene-17β-carboximide of the formula III can be prepared from it's respective acid chloride.

### DESCRIPTION OF THE INVENTION

The process for the preparation of Dutasteride, which comprises;
(i) reacting 3-oxo-4-aza-5α-androst-1-ene-17β-carboximide with 2-Iodo 1,4-bis (trifluoromethyl) benzene in the presence of organic solvents like aromatic hydrocarbon solvents like toluene, xylene or dimethylformamide or dimethylsulfoxide and a base comprising of potassium carbonate, sodium carbonate, sodium bicarbonate and metal halides like copper halides under heating conditions till the reaction completes;
(ii) cooling the reaction mass of step (i) to 10-35° C. with stirring;
(iii) filtration of the separated solid from step (ii) by conventional methods;
(iv) the wet solid obtained from step (iii) in an organic solvents like ethyl acetate or halogenated hydrocarbon solvents like dichloromethane, dichloro ethane or aromatic hydrocarbon solvents like toluene, xylene;
(v) heating the reaction mass of the step (iv) up to reflux for about 30 minutes;
(vi) filtration of the reaction mass of step (v) at reflux temperature;
(vii) washing the filtrate of step (vi) with 2-10% of hydrochloric acid solution;
(viii) washing the organic solution of step (vii) with 5-10% of basic solution;
(ix) finally washing the organic solution of step (viii) with water;
(x) distillation of the solvent up to 70-75% from organic solution of step (ix) under reduced pressure;
(xi) cooling the reaction solution of step (x) to 10-35° C. with stirring;
(xii) filtration of separated solid of step (xi) by conventional methods;
(xiii) dissolution of the solid of step (xii) in the mixture of tetrahydrofuran (THF) and water at reflux temperature;
(xiv) cooling the reaction solution of step (xiii) to 25-35° C. for 2 hours;
(xv) filtration of the separated solid of step (xiv);
(xvi) dissolving the wet solid of step (xv) in methanol;
(xvii) distilling off the methanol from step(xvi) to minimum volume under reduced pressure;
(xviii) isolation of the solid from the residue of step (xvii) with ethyl acetate;

The process of the present invention, according to the above said process of step (i) wherein the reaction can also be carried out with or without solvent like Dimethyl formamide, Dimethyl sulfoxide, orthoxylene, N-methylpyrolidinone.

The process of the present invention, according to the above said process of step (i) wherein the reaction can be carried out using of copper powder, copper iodide, copper chloride, copper bromide.

The process of the present invention, according to the above said process of step (i) wherein the reaction can be carried out using of sodium carbonate or potassium carbonate or sodium bicarbonate or mixtures thereof.

The process of the present invention, according to the above said process of step (v) wherein the product can be isolated using preferably ethyl acetate.

Another embodiment of the present invention was preparation of 3-oxo-4-aza-5α-androst-1-ene-17β-carboximide, which involves reaction between 3-oxo-4-aza-5α-androst-1-ene-17β-carboxylic acid of Formula II, thionyl chloride, pyridine and ammonia gas. The synthetic scheme represented as bellow (Scheme 4)

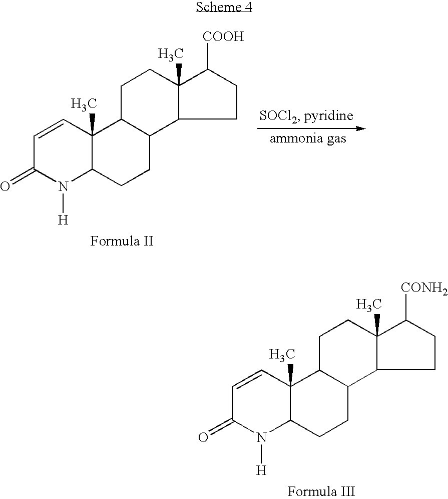

Process for the preparation of compound of formula III comprises:
i) stirring the mixture of 3-oxo-4-aza-5α-androst-1-ene-17β-carboxylic acid, of Formula II toluene, pyridine and thionyl chloride (SOCl₂) at 25-35° C. for about 2-3 hours;
ii) after the completion of the reaction of step (i) treated with source of ammonia for about 8-10 hours;
iii) filtration of the separated solid from the reaction mass of step (ii) and wash the solid with water;
iv) purification of the obtained solid in step (iii) in a suitable solvent and mixture thereof.

Another embodiment of the present invention was preparation 2-Iodo-1,4-bis(trifluoromethyl) benzene of Formula IV, which involves reaction between 2, 5-bis(trifluoro methyl) aniline of Formula IVa, sodium nitrite, hydrochloric acid and potassium iodide, which can be represented by Scheme 5

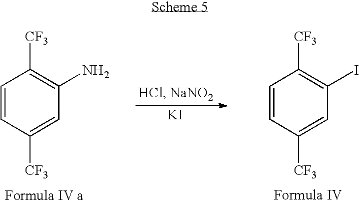

Process for the preparation of compound of Formula IV comprises:
i) stirring the reaction mixture of hydrochloric acid, water, amino compound of formula IVa, sodium nitrite and potassium iodide at 0-5° C. for about 10-45 minutes;
ii) maintaining the above reaction mass of step (i) at 60-80° C. for about 2-3 hours;
iii) after completion of the reaction of step (ii), extracting the product with halogenated hydrocarbon solvents like dichloromethane, dichloroethane and chloroform;
iv) distillation of the organic solvent of step (iii) under reduced temperature;
v) isolation of the required compound from the residue of step (iv);

The following examples are only illustrative and are not intended to limit the scope of the invention.

### EXAMPLE 1

### PREPARATION OF 3-OXO-4-AZA-5α-ANDROST-1-ENE-17β-CARBOXIMIDE (FORMULA III)

3-Oxo-4-aza-5α-androst-ene-17β-carboxylic acid (50 gram) and toluene (750 ml) were mixed together and heated at azeotropic reflux condition for 30 to 60 minutes. (Trace amount of water was removed azeotropically). The resulting solution was cooled to 25 to 35 degree C. under nitrogen atmosphere. Pyridine (6.3 ml) was added to the cooled solution, which was then stirred for about 15 minutes. Then, thionyl chloride (14.0 ml) was added slowly for over 20 minutes. The resulting reaction mixture was maintained at 25-35° C. temperature for about 2-3 hours and then ammonia gas was passed through the reaction mixture till the reaction was completed (8 to 10 hrs). After the completion of the reaction mixture was filtered and washed with toluene (100 ml). The resulting compound was dried for 1-2 hours. The resultant wet material was slurried in water (500 ml) for about 2 hours. Filtered the solid and washed with water (50.0 ml) to get the reaction mass pH up to 6.5 to 7.5. The filtered compound was dried at 70-75° C.

### EXAMPLE 2

### PURIFICATION OF 3-OXO-4-AZA-5α-ANDROST-1-ENE-17β CARBOXIMIDE

3-oxo-4-aza-5α-androst-1-ene-17β-carboximide (50.0 gram) was dissolved in a mixture of methylene chloride and methanol in the ratio of 8:2 (2250 ml), which was stirred for about 30 minutes at 25-35° C. Insoluble impurities were removed by a filtration with help of a washing solution, 2% aqueous sodium hydroxide solution (3×63.0 ml). 70-80% the solvent was distilled off at below 50° C. under reduced pressure. Methanol (150.0 ml) was added to the reaction mass and distilled off 50-60% of the solvent under reduced pressure. After the reaction mass was stirred for 10-20 minutes at 25-35° C. solids were filtered, washed with methanol (100.0 ml), and dried at 60-70° C.

### EXAMPLE 3

### PREPARATION OF 3-OXO-4-AZA-5α-ANDROST-1-ENE-17β CARBOXYLIC ACID (FORMULA II)

2,3-dichloro-5,6-dicyano-1,4-benzoquinone (85.3 gr) and dry toluene (1500.0 ml) were charged in a reactor and heated to azeotropic reflux condition for about 2 hours. (Trace amount of water was removed azeotropically). The refluxed reaction mixture was cooled to 25-35° C. 3-Oxo-4-aza-5α-androstane-17β-carboxylic acid (100 gr), bis (trimethylsilyl) trifluoroacetamide (322.2 gr) and triflic acid (1.6 ml) were added into the cool reaction mixture under nitrogen atmosphere at 25-35° C., and then the resulting reaction mixture was heated to 105-108° C. for about 20-24 hours under nitrogen atmosphere. After the reaction mixture was cooled to 50-60° C., water (250 ml) was added, and the resultant solid was filtered. Wet material was then taken into water (100 ml), heated to 50-60° C., and stirred for about 30 minutes. The solids were filtered under reduced pressure for 1-2 hours. This hot water slurry (500 ml) and filtering process was repeated, and the filtered solids were washed the solid with water (100 ml). Wet solids were added into acetone (500 ml), which was then heated to reflux temperature for 20-30 minutes. The resulting reaction mixture was cooled to 25-35° C. and maintained for 30-45 minutes. The solids were filtered under reduced pressure and washed with acetone (50.0 ml). The filtered wet compound was added into a mixture of dichloromethane and methanol solution (600 ml), in the ratio of 8:2, which was stirred at 25-35° C. for 20-30 minutes. Then 80% of the solvent was distilled off under reduced pressure. After cooling the reaction mass to 25-35° C., methanol (300 ml) was added. The solvent (approximately to 100 ml) was again distilled off under reduced pressure. The condensed reaction mixture was cooled to 25-35° C. and maintained for about 30 minutes. Solids were filtered and washed with methanol (100 ml). The filtered solids were dried at 60-70° C. under reduced pressure, to get the title compound (55 gram, purity 99.5%).

### EXAMPLE 4

### PREPARATION OF 2-IODO-1,4-BIS(TRIFLUOROMETHYL) BENZENE (FORMULA IV)

Water (250 ml) and hydrochloric acid (2385 ml) were charged in a reactor and cooled to 0-5° C. 2,5-Bis (trifluoro methyl) aniline (250 gr) was added to the cooled aqueous HCl at about 0-5° C., and the resulting solution was stirred for 10 minutes. Slowly a solution of sodium nitrite (94.1 gr) in water (415 ml) was added to the aniline solution over 30 minutes at 0-5° C. The reaction mixture was stirred for 10 minutes at the same temperature followed by addition of a solution of potassium iodide (225 grams) in water (585 ml) to the reaction mixture for 30 minutes. After heating the resulting reaction mixture to 60-70° C. for about 3-4 hours, it was cooled to 25-35° C. and was extracted two times with methylene chloride (250 ml×2). The combined organic layer was washed with 5% sodium bicarbonate solution (2×250 ml) followed by 5% sodium thiosulfate solution (2×250 ml). Finally, the organic layer was washed with water (2×250 ml), and the organic solvent was distilled off under reduced pressure to get the title compound. Yield: 420.0 gr, Purity: 98.0%

### EXAMPLE 5

### PREPARATION OF 17β-N-[2,5-BIS (TRIFLUOROMETHYL) PHENYL]CARBAMOYL-4-AZA-5-α-ANDROST-1-EN-3-ONE (DUTASTERIDE)

Potassium carbonate (11 grams) and xylene (125 ml) were heated to azeotropic reflux temperature for 2 hours to remove water azeotropically. The xylene mixture was cooled to about 30-40° C., to which 3-oxo-4-aza-5α-androst-1-ene-17β-carboximide (25 grams), copper powder (15.1 gram) and 2-Iodo-1,4-bis (trifluoromethyl) benzene (81 grams) were added. Then, the resulting mixture was heated to 140-150° C. for about 50-60 hours. After cooling the reaction mixture to 25-35° C. and maintaining for about 3-4 hours at the same temperature, washed the solid with o-xylene (12.5 ml), and dried for 2 hours under reduced pressure. The dried solid was added to ethyl acetate (750 ml), which was then heated to reflux for 30 minutes. The refluxed acetate solution was filtered to remove in soluble impurities with help of additional 500 ml of ethyl acetate. The filtrate was washed with aqueous hydrochloric acid solution (12.5 ml of HCl in water 112.5 ml) at 60-65° C., and subsequently with 5% aqueous sodium bicarbonate solution (3×125 ml) followed by water (2×125 ml) at 50-60° C. 70-75% of the solvent was distilled off under reduced pressure and the remaining solution was cooled to 25-35° C. Solids were separated, filtered, and washed the solid with ethyl acetate (25 ml), and dried the solid at 65-70° C. under reduced pressure. The isolated solids were added to a (1:1) mixture of tetrahydrofuron and water solution (375 ml) which was then heated to reflux for dissolution. After refluxing the solution, for 15 minutes, the solution was filtered while it is still hot. The filtrate was cooled to 25-35° C. and maintained for 2 hours. Precipitated solids were filtered, washed with a (1:1) mixture of tetrahydrofuron and water solution (25 ml), and dissolved in methanol (175 ml) followed by carbon treatment (1.2 grams). The methanol was distilled off up to 90% of its initial volume. Ethyl acetate (100 ml) was added to the condensed methanol solution. The solvent of the resulting solution was distilled off again until the total volume reached 25% of the original volume. Condensed solution was cooled to 25-35° C. and maintain for 2 hours for separation of solids. Filtered the reaction mass and washed the solid with ethyl acetate (25 ml), dried the solid at 65-70° under reduced pressure for 15-30 hours, to get the title compound. (Yield: 15.0 gr, Purity: 99.8%).

1. The novel and simple process for the preparation of Dutasteride, which comprises:
(i) reacting 3-oxo-4-aza-5α-androst-1-ene-17β-carboximide with 2-Iodo 1,4-bis (trifluoromethyl) benzene in the presence of organic solvent comprises of aromatic hydrocarbon solvents like toluene, xylene or dimethylformamide or dimethylsulfoxide and a base comprising of potassium carbonate, sodium carbonate, sodium bicarbonate and metal halides preferably copper halides under heating conditions till the reaction completes;
(ii) cooling the reaction mass of step (i) to 10-35° C., preferably 10-25° C. under stirring;
(iii) filtration of the separated solid from step (ii) by conventional methods;
(iv) the wet solid obtained from step (iii) in an organic solvents like ethyl acetate or halogenated hydrocarbon solvents like dichloromethane, dichloro ethane or aromatic hydrocarbon solvents like toluene, xylene;
(v) heating the reaction mass of the step (iv) up to reflux for about 30 minutes;
(vi) filtration of the reaction mass of step (v) at reflux temperature;
(vii) washing the filtrate of step (vi) with 2-10% of hydrochloric acid solution;
(viii) washing the organic solution of step (vii) with 5-10% of basic solution;
(ix) finally washing the organic solution of step (viii) with water;
(x) distillation of the solvent up to 70-75% from organic solution of step (ix) under reduced pressure;
(xi) cooling the reaction solution of step (x) to 10-35° C. under stirring;
(xii) filtration of separated solid of step (xi) by conventional methods;
(xiii) dissolution of the solid of step (xii) in the mixture of tetrahydro furan and water at reflux temperature;
(xiv) cooling the reaction solution of step (xiii) to 25-35° C. for 2 hours;
(xv) filtration of the separated solid of step (xiv);
(xvi) dissolving the wet solid of step (xv) in methanol;
(xvii) distillation off the methanol from step (xvi) to minimum volume under reduced pressure;
(xviii) isolation of the solid from the residue of step (xvii) with ethyl acetate.

2. The process according to the claim 1 of step (i) wherein the reaction can be carried out in dimethylformamide, dimethylsulfoxide, xylene, N-methylpyrolidinone or neat reaction (without any solvent), preferably xylene.

3. The process according to the claim 1 of step (iv) wherein the organic solvent is preferably ethylacetate.

4. The process according to the claim 1 of step (i) wherein the reaction can be carried out at a temperature of about 120-170° C., preferably 140-145° C.

5. The process according to the claim 4, wherein the reaction can be carried out under positive pressure conditions.

6. The process for the preparation of 3-oxo-4-aza-5α-androst-1-ene-17β-carboximide by the reaction between 3-oxo-4-aza-5α-androst-1ene-17β-carboxylic acid and thionyl chloride, the presence of pyridine and ammonia gas.

7. The process according to claim 6, which comprises:
(i) stirring the mixture of 3-oxo-4-aza-5α-androstane-17β-carboxylic acid, toluene, pyridine and thionyl chloride at 25-35° C. till to the reaction completes;
(ii) after the completion of the reaction of step (i) treated with source of ammonia till to the completion of the reaction;
(iii) filtration of the separated solid from the reaction mass of step (ii) and wash the solid with water medium till pH reaches to 6.5 to 7.5;

8. The process for the preparation of 2-Iodo-1,4-bis(trifluoromethyl) benzene by the reaction between 2,5-bis(trifluoro methyl) aniline and sodium nitrite, hydrochloric acid and potassium iodide.

9. The process according to claim 8 which comprises:
(i) stirring the reaction mixture of hydrochloric acid, water, 2,5-Bis(trifluoro methyl) aniline, sodium nitrite and potassium iodide at 0-5° C.;
(ii) maintaining the above reaction mass of step (i) at 60-80° C.;
(iii) after completion of the reaction of step (ii), extracting the product with halogenated hydrocarbon solvents;
(iv) distillation of the organic solvent of step (iii);
(v) isolation of the product of step (iv) under high vacuum distillation.

10. A compound 3-oxo-4-aza-5α-androst-1-ene-17β-carboximide, which can be used as an intermediate for the preparation of dutasteride.

11. A compound 2-halo-1,4-bis(trifluoromethyl) benzene, which can be used as an intermediate for the preparation of dutasteride.

12. Pharmaceutical composition consists of 17β-N-[2,5-bis (trifluoromethyl) phenyl] carbamoyl-4-aza-5-α-androst-1-en-3-one (Dutasteride) obtained by the process according to claim 1 as an active ingredient.
